(19) United States
(12) Patent Application Publication
(10) Pub. No.: US 2012/0238007 A1
(43) Pub. Date: Sep. 20, 2012

(54) Culture Bottles with Internal Sensors

(75) Inventors: Mark S. Wilson, Hillsborough, NC (US); Ronnie J. Robinson, St. Charles, MO (US); Christopher S. Ronsick, Durham, NC (US); Patrick Yerbic, St. Louis, MO (US); Mark J. Fanning, Florissant, MO (US); Stanley M. Philipak, Augusta, MO (US)

(73) Assignee: BIOMERIEUX, INC., Durham, NC (US)

(21) Appl. No.: 13/193,243

(22) Filed: Jul. 28, 2011

Related U.S. Application Data
(60) Provisional application No. 61/400,634, filed on Jul. 29, 2010.

(51) Int. Cl. C12M 1/24; B05D 7/22

(52) U.S. Cl. 435/288.1; 427/230

(57) ABSTRACT

Specimen containers incorporating a sensor are provided with features for decreasing the volume of polymer matrix material required for the sensor. Such volume-reducing features can take the form of scallop-like indentations projecting inwards towards the interior of the container formed in the transition between the side wall of the container and the base of the container. Alternatively, the base of the container includes a raised rim extending upwards into the interior of the body inward of and spaced from the side wall. The rim defines a chamber for the sensor. Methods of manufacturing specimen containers with cured liquid phase sensor matrix materials are also disclosed.

### CROSS REFERENCE TO RELATED APPLICATION

This application claims the benefit of U.S. Provisional Patent Application No. 61/400,634, entitled “Culture Bottles with Internal Sensor”, filed Jul. 29, 2010, which is incorporated herein.

### BACKGROUND

1. Field

This invention relates to bottles for culturing blood or other biological specimens.

2. Description of Related Art

Blood culture bottles are known in the art and described in the patent literature, see, e.g., U.S. Pat. Nos. 4,945,060; 5,094,955; 5,162,229, 5,217,876, 4,827,944; 5,000,804; 7,211,430 and US 2005/0037165. Analytical instruments for analyzing the bottles for presence of organisms include U.S. Pat. Nos. 4,945,060; 5,094,955; 6,709,857 and 5,770,394, and WO 94/26874.

Blood culture bottles having an internal colorimetric sensor for detecting microbial growth within the culture bottle are described in U.S. Pat. Nos. 4,945,060, 5,094,955, 5,162,229 and 5,217,876. The sensor is located in the interior of the bottle at the bottom or base of the bottle. Increased concentration of CO₂ within the bottle as a byproduct of microbial growth causes the sensor to change color. The change in color is detected by a photodetector in an associated analytical instrument.

The colorimetric sensor used in such bottles can be made from a polymer matrix. The polymer matrix can be poured into the base of the bottle in which they flow to a uniform level. The polymer matrix is cured (solidified) by radiation or heat.

Other blood culture bottles are known in the art which use fluorescence sensors for determining microbial growth, including the BACTEC™ bottles produced by Becton Dickinson.

### SUMMARY

The present inventors have appreciated that the instrument interrogating the colorimetric sensor in the bottles of type shown in U.S. Pat. Nos. 5,162,229 and 5,217,876 uses a light source which may impinge only a small part of the colorimetric sensor and not the entire base of the bottle. The present designs provide for bottle configurations which take advantage of this insight by reducing the amount of the sensor polymer matrix material required to make a functioning colorimetric sensor, thereby reducing the cost of the bottle. The designs achieve this reduction in the volume of polymer matrix sensor material by providing novel constructions of the bottle. The techniques are also applicable to other types of sensors placed within culture bottles, including the fluorescence sensors of the BACTEC™ bottles and the like.

In one aspect, a specimen container for receiving a sample is described having a bottle-like body with a side wall defining an interior of the body, an upper portion and base. The side wall includes a transition portion connecting the side wall to the base. The transition portion features a plurality of scallops in the form of indentations in the side wall. The scallops are formed circumferentially around the transition portion and extend inwardly toward the interior of the container so as to reduce the volume thereof. A wide variety of scallop designs are possible to achieve this result, several of which are shown in the appended drawings by way of example. A sensor (e.g., colorimetric or fluorescence sensor) is positioned in the interior of the body in the transition portion.

In some embodiments the bottle-like body is cylindrical in form, however this is not critical and the volume-reducing features of this disclosure can be formed in bottles with other configurations, e.g., square bottles.

In yet another aspect, a method of manufacturing a specimen container is described, comprising the steps of providing a bottle with the above-described scallop features, and introducing a liquid phase sensor polymer matrix into the reduced volume region defined by the plurality of scallops and curing the polymer matrix into a solid phase in place.

In another aspect, a specimen container for receiving a sample is provided, comprising a bottle-like body having a side wall defining an interior of the body, a top portion and a base, and a sensor positioned within the specimen container. The base may include a raised rim extending upwards from the base into the interior of the body inward of and spaced from the side wall. The rim defines an interior chamber and exterior chamber within the bottle. In one embodiment, the interior chamber may contain the sensor (e.g., colorimetric or fluorescence sensor). In another embodiment, the exterior chamber may contain the sensor.

Again, in some embodiments the side wall of the body is cylindrical and the raised rim may be circular and centered on the central axis of the body. However, the body may take other shapes, such as a square-like shape. Also, the raised rim may take other shapes, such as a square, oval or other shape.

In another aspect, a method of manufacturing a specimen container is provided, comprising the steps of: providing a bottle-like body as described above having a raised rim extending upwards from the base defining interior and exterior regions or chambers at the base, and introducing a liquid phase sensor polymer matrix (or sensor) into either the interior or exterior chamber defined by the raised rim and curing the polymer matrix (or sensor) into a solid phase in place.

### BRIEF DESCRIPTION OF THE DRAWINGS

FIG. 1 is a perspective view of a specimen container having reduced-volume features proximate to the base of the container in order to reduce the volume of the container proximate to the base.

FIG. 2 is a more detailed view of the scallop features of FIG. 1.

FIGS. 3-5 are cross-sectional views of the container of FIG. 1.

FIG. 6A is a plan view of the bottom portion of a specimen container showing an alternative arrangement of the scallops of FIG. 1.

FIG. 6B is a cross-sectional views of the container of FIG. 6A.

FIG. 7 is a perspective view of an alternative configuration of the specimen container, partially broken away, showing a raised rim projecting upwards from the base of the container defining a chamber for receiving the sensor matrix material.

FIGS. 8A-8D are cross-sectional views of different embodiments of the container in FIG. 7.

FIG. 9A is a plan view of the colorimetric sensor used in the embodiment of FIG. 1, shown isolated from the container, with the savings in volume indicated by the area out-side of the star-shaped colorimetric sensor.

FIG. 9B is a plan view of the colorimetric sensor used in the embodiment of FIG. 7, with the savings in volume indicated by the area outside of the circular sensor.

FIG. 10 is a cross-section showing the embodiment of FIG. 7 proximate to a detector for detecting the color change in the colorimetric sensor due to microbial growth.

FIG. 11 is a side elevation view of the container of FIG. 1 showing a detent ring formed in the lower portion of the container.

### DETAILED DESCRIPTION

Specimen containers are described herein which include features for reducing the volume of polymer matrix material needed to form a sensor incorporated into the interior of the container. In one example, the specimen container is in the form of a culture bottle used for culturing a biological sample such as, for example, blood.

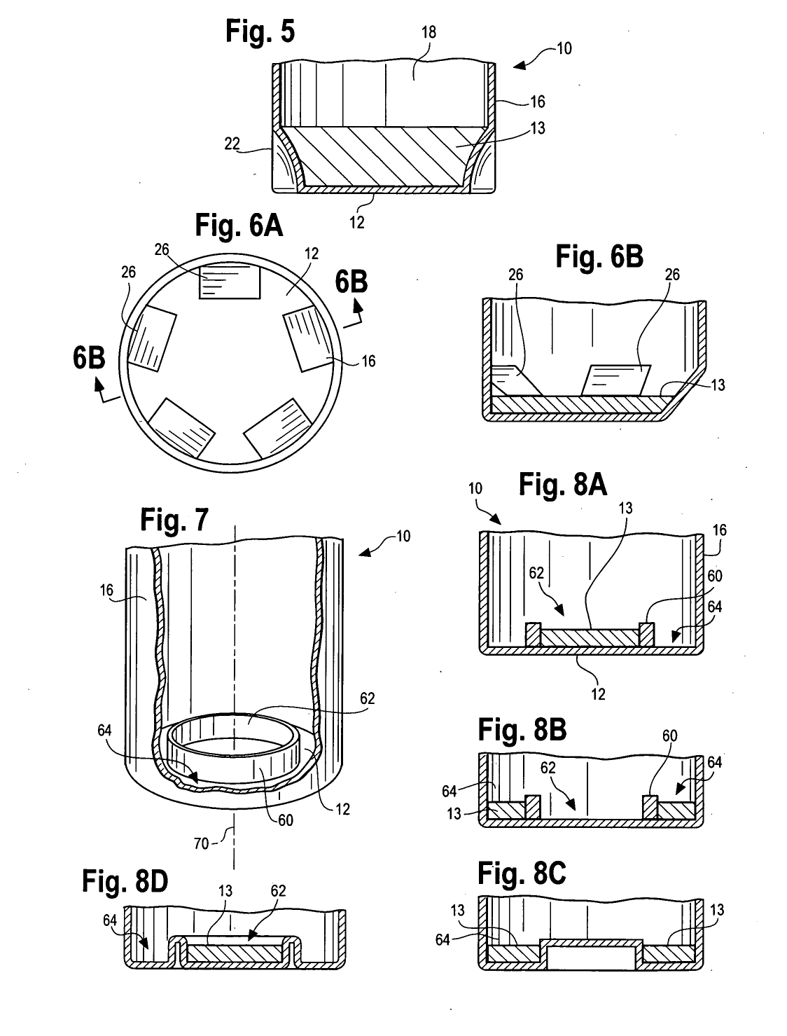

Referring now to FIGS. 1-5, a first embodiment of specimen container 10 having reduced volume of sensor material will be described. The container 10 includes a base 12. The container 10 has reduced-volume features proximate to the base 12 in order to reduce the volume of sensor material 13 (shown in FIG. 5) functioning as the colorimetric or fluorescence or other type of sensor for the bottle. The container 10 is in the form of a bottle-like body 14 having a cylindrical side wall 16 defining an interior 18. The container includes an upper portion 20, the configuration of which is not particularly important. The upper portion 20 is typically sealed with a stopper, closure, septum or other closure element (not shown). The cylindrical side wall 16 has a transition portion 22 at the lower portion thereof connecting the side wall 16 to the base 12. While the body 14 is shown in the form of a cylinder this is not particularly critical and the body can take other forms, such as a square bottle.

In one possible embodiment the container 10 is blow molded or injection blow molded from a plastic material. The container 10 can be monolayer or multilayer plastic bottle, as is well known in the art. Alternatively, the container 10 can be fabricated from glass. The manner of forming the container per se is not particularly important. In one form, the side wall 16, transition portion 22 and base 12 are integral (i.e., the container is made in one piece). In alternative configurations the bottle could be made from two separate pieces, one forming the side wall 16 and the other forming the transition portion 22 and base 12; the two pieces could be joined together e.g. by sonic welding, adhesive, or other means.

As shown in FIGS. 1-4, the transition portion 22 includes a plurality of scallops 26. The term “scallops” is meant to refer to indentations in the cylindrical side wall 16. The scallops 26 are formed circumferentially around at least a portion of the transition portion 22, and in some embodiments are formed completely around the periphery or circumference of the base 12. The scallops extend or project inwardly toward the interior of the container 10 as indicated in FIGS. 1-5 to reduce the volume of the container (i.e., reduce the volume of the container as compared to what it would otherwise be without the scallops, that is if cylindrical shape of the side wall 16 continued to the base 12). In one embodiment, the scallops reduce the volume at the base 12 of the container 10 and in particular reduces the volume of sensor polymer matrix material needed to form the colorimetric or fluorescence sensor in the container. The transition portion 22 includes at least 2 scallops formed circumferentially around the periphery or circumference of the base 12. Typically, the transition portion 22 will include from about 3 to about 16 scallops, from about 4 to about 12 scallops, or from about 5 to about 10 scallops. As shown in FIGS. 1-5 and 9A, the transition portion 22 contains 8 scallops. The present inventors have unexpectedly found that the presence of the scallops at the base of the container 10 adds more strength and rigidity to the container compared to traditional containers that do not have scallops. The additional strength and rigidity of the scalloped base will also reduce any distortion or distention of the base that may otherwise occur through the autoclave cycle. If the base distends through the autoclave cycle, then the bottle may tend to wobble.

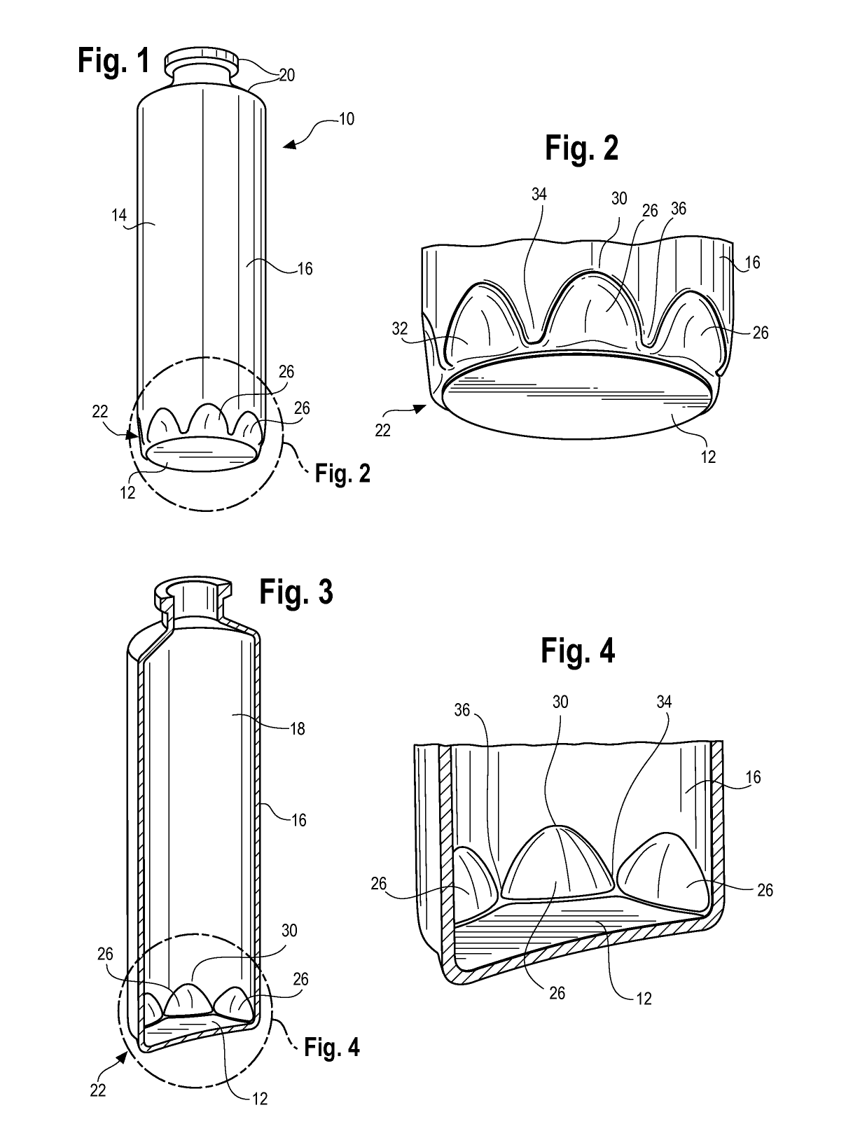

The scallops 26 can take a wide variety of forms and be spatially arranged around the base of the bottle 10 in a variety of configurations. No particular form is critical. In one form, the scallops are arcuate-like indentations shown in FIGS. 1-4 having an apex 30 oriented in the direction of the top portion of the container and a bottom 32 portion oriented towards the base 12 of the container 10. The bottom portion 32 has two opposed corners 34 and 36 (FIG. 4). The scallops 26 are positioned about the transition of the container such that the corners 34 and 36 of each of the scallops is adjacent to a corner of another one of the scallops, as shown in FIG. 2. Thus, the scallops are circumferentially spaced around the bottom of the bottle adjacent to one another. Non-symmetric placement of the scallops 26 are also possible and the scallops need not all be of the same size or shape. Additionally, the scallops could be spaced from each other.

The base 12 as shown in FIGS. 3 and 4 may have a very slight inward deflection or dome-shape (also known in the art as “push-up”) in order to prevent the center of the exterior surface of the base 12 from getting dirty or being scuffed as the bottles move along a conveyor belt. In addition, by virtue of the dome the center 12 will not distend and make for a wobbling bottle when the bottle is pressurized, as in autoclaving. The presence of the dome may add additional strength to the bottle and increase the stability of the bottle, i.e., reduces the tendency of the bottles to wobble. The polymer matrix material forming the sensor 13 of FIG. 5 is initially in a liquid phase and inserted (e.g., poured) into the base of the container 10 and cured in place, e.g., using heat, radiation or other technique.

FIG. 6A is a perspective view of the bottom portion of a specimen container of FIG. 1 showing an alternative arrangement of the scallops 26, as seen from the interior of the container. As shown in FIGS. 6A-B, the scallops may be in the form of ramp-like indentations that are spaced from each other extending around the periphery of the transition portion.

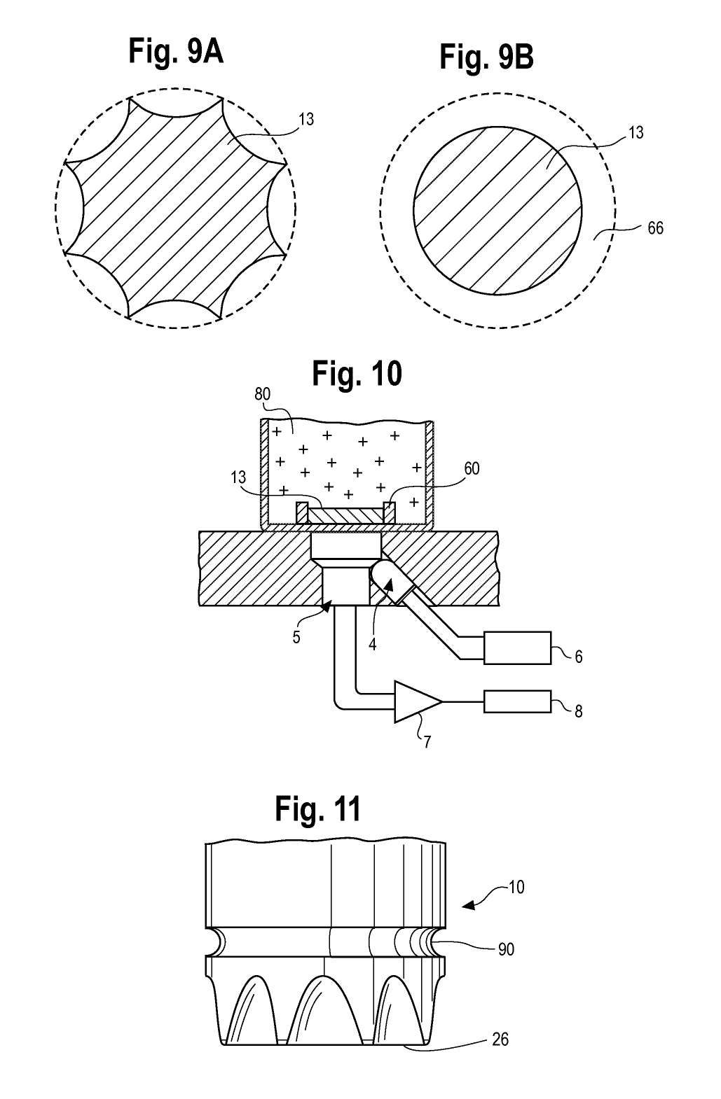

The feature of the scallops 26 projecting inwardly into the interior of the container operates to reduce the volume at the base 12 of the container 10 and in particular reduces the volume of sensor polymer matrix material needed to form the colorimetric or fluorescence sensor in the container. This is shown, for example, in FIG. 9A, with the volume of the colorimetric sensor 13 is reduced by from about 10 to about 20 percent compared to conventional specimen containers. The volume saved is indicated by the area outside of the periphery of the colorimetric sensor 13. In some configurations, the volume of sensor 13 is reduced by about 5 to about 50 percent, from about 10 to about 40 percent, or from about 10 to about 30 percent compared to conventional specimen containers.

FIG. 7 is a perspective view of a second embodiment of specimen container 10, shown partially broken away to illustrate a raised rim 60 projecting upwards from the base 12 of the container 10. As shown, the rim 60 is spaced from the cylindrical wall 16 of the container 10. The rim 60 forms an interior chamber 62 and an exterior chamber 64. As shown in FIG. 7, the interior chamber 62 may receive the polymer matrix or sensor 13, thereby reducing the volume of sensor material needed compared to a conventional specimen container (i.e., a specimen container not containing a raised rim). FIG. 8A shows a cross-sectional view of the bottom portion of the container of FIG. 7 showing the interior chamber 63 filled with a polymer matrix or sensor 13. In another embodiment, the exterior chamber 64 may receive the polymer matrix or sensor 13 (see, e.g., FIG. 8B). In yet another embodiment, the base of the container may contain an indentation rising up from the base 12 that creates an exterior chamber 64 for containing a reduced volume of polymer matrix or sensor, as shown for example in FIG. 8C. In still another embodiment, the rim 60 may be formed as an indented ring 90, where the entire ring structure is formed as an indentation in the base 12 of the container 10, creating interior and exterior chambers 62, 64, as shown for example in FIG. 8D. As shown in FIG. 8D the interior chamber 62 can be filled with polymer matrix or sensor 13. However, alternatively, as described elsewhere herein the exterior chamber 64 can receive the polymer matrix or sensor 13. As previously described, the polymer matrix material forming the sensor is typically inserted (e.g., poured) into the interior or exterior chamber 62, 64 in a liquid phase and cured in place, e.g., using heat, radiation or other technique.

The reduced diameter of the rim 60 of FIGS. 7 and 8A-8D operate to reduce the volume of the polymer matrix material needed to form the colorimetric sensor 13. For example, the diameter of the rim 60 shown in FIGS. 7 and 8A may be between 50 and 90 percent of the diameter of the cylindrical side wall 16 of the container And may reduce the volume of polymer matrix or sensor 13 by from about 5 to about 50 percent, from about 10 to about 40 percent, or from about 10 to about 30 percent compared to conventional specimen containers. This reduction in volume is indicated in FIG. 9B, with the material saved being the area 66 outside of the colorimetric sensor 13. Similarly, in other embodiments (for example, those shown in FIGS. 8B-8D), the volume of polymer matrix or sensor 13 needed may also be reduced by from about 5 to about 50 percent, from about 10 to about 40 percent, or from about 10 to about 30 percent compared to conventional specimen containers.

As shown in FIG. 7, the raised rim 60 is preferably centered on the central axis 70 of the container. This insures rotational symmetry in the bottle, meaning that the bottle need not be inserted into the detection instrument in a particular orientation in order for optical interrogation of the bottle to occur successfully. Alternatively, it would be possible to form the reduced volume features of FIGS. 1 and 7 in a non-rotationally symmetric manner, such as by centering the rim 60 not on the axis 70 but rather to one side, and including features in the bottle and/or holding structure that require the bottle to be inserted into the detection instrument in a particular orientation, so that the colorimetric sensor is correctly positioned relative to the light source and photodetector (or other detection instrumentation for monitoring the sensor 13). Similarly, the scallops 26 of FIGS. 1-6 could be oriented such that more reduction in volume occurs on one side of the transition portion and less, or no, reduction in volume occurs on the other side of the transition portion.

FIG. 10 shows the container of FIG. 1 with a colorimetric sensor placed in the specimen container in the presence of a sample 80. FIG. 10 also shows the detection instrumentation for colorimetric sensors, namely a light source 4 and a photodetector 5, and the associated electronics including a current source 6, current to voltage converter 7 and a low pass filter 8. These details are described in the patent literature and therefore a detailed discussion is omitted.

FIG. 11 is a side elevation view of the container of FIG. 1 with an optional detent ring or indentation 90 extending around the circumference of the bottle 10 in the lower portion thereof above the scallops 26. The detent ring 90 cooperates with an optional holding structure (not shown) that may be used in the arrangement of FIG. 10 for holding the bottle in position shown in FIG. 10. Such holding structure could include an elastomeric protrusion or raided bead that fits into the detent ring or indentation 90 to correctly position the bottle 10 immediately adjacent to the detection instrumentation of FIG. 10. The detent ring 90 also may serve to prevent the bottle from moving in the holding structure during agitation of the bottle or tilting of the bottle below horizontal e.g., for sampling of the bottle 10 or as part of an agitation regime. In this respect, teachings of FIG. 9 of PCT publication WO 94/26974 may be adapted for use with the detent ring 90 of present bottle. The content of WO 94/26974 is incorporated by reference herein. The detent ring 90 may of course be used with any of the other bottle designs of this disclosure including the embodiment of FIGS. 7 and 8. The detent could also take the form of raised surface, e.g., bead extending around the cylindrical side wall. In another embodiment, the detent ring or indentation 90 may be located substantially at the base 12 of the container 10 resulting in a reduced diameter at the base 12 and reduced volume of polymer material or sensor 13.

In another aspect, a method of manufacturing a specimen container for receiving a sample includes the steps of providing a bottle-like body 14 having a side wall 16 defining an interior 18 of the body, an upper portion 20 and base 12, the side wall 16 including a transition portion 22 connecting the side wall to the base, wherein the transition portion comprises a plurality of scallops 26 (FIGS. 1-6) comprising indentations in the side wall 16, the plurality of scallops 26 extending at least partially around the transition portion and extending inwardly toward the interior of the container to reduce the volume thereof; and introducing a liquid phase sensor polymer matrix 13 into the reduced volume region defined by the plurality of scallops (see FIG. 5) and curing the polymer matrix into a solid phase in place.

In a further aspect, a method of manufacturing a specimen container includes the steps of: providing a bottle-like body (FIG. 7) having a side wall 16 defining an interior of the body, an upper portion and a base, wherein the base further comprises a raised rim 60 extending upwards into the interior of the body, the rim defining a chamber 62; and introducing (e.g., pouring, optionally with the aid of a nozzle or other apparatus) a liquid phase sensor polymer matrix 13 into the interior or exterior chamber 62, 64 defined by the raised rim 60 (see FIG. 8A) and curing the polymer matrix into a solid phase in place, e.g. with heat. In other embodiment, the raised rim 60 may be formed in the base 12 of the container 10 as an inward indent formed in and projecting upwards from the base 12, as shown in FIGS. 8C. In still another embodiment, the base 12 of the container 10 may contain a disk-shaped indented formed in and projecting upwards from the base, as shown in FIG. 8D.

The container 10 is loaded with a culture medium (not shown) at the time of manufacture. At the time of use, a sample (FIG. 10, 80) is introduced into the container and the container subject to incubation in order to foster growth of microbial agent in the sample due to the presence of the culture medium. The colorimetric sensor 13 is periodically interrogated by the detection instrument of FIG. 10 to determine whether microbial growth has occurred by means of detecting a color change in the colorimetric sensor. These aspects are known in the patent literature and therefore a detailed discussion is omitted for the sake of brevity.

The materials for the colorimetric sensor are also described in the patent literature and therefore a description is omitted for the sake of brevity. See, e.g., U.S. Pat. No. 5,094,955, the content of which is incorporated by reference herein. Fluorescence sensors are also described in the patent literature, see e.g., U.S. Pat. No. 6,989,246, which is also incorporated by reference herein.

Variation from the specifics of the disclosed embodiments are of course possible and will be apparent to persons skilled in the art without departure from the scope of the invention. All questions concerning scope are to be answered by reference to the appended claims. The appended claims are offered as further descriptions of the disclosed inventions.

We claim:

1. A specimen container for receiving a sample, comprising:
a body having a side wall defining an interior of the body, an upper portion and a base, the side wall including a transition portion connecting the side wall to the base,
wherein the transition portion comprises a plurality of scallops providing indentations in the side wall, the plurality of scallops extending circumferentially at least partially around the transition portion and extending inwardly toward the interior of the container to reduce the volume thereof; and
a sensor in the interior of the body in the transition portion.

2. The specimen container of claim 1, wherein the scallops comprise arcuate-like indentations having an apex oriented in the direction of the top portion of the container and a bottom portion oriented towards the base of the container, the bottom portion having two opposed corners, and wherein the corners of each of the plurality of scallops in the transition portion is adjacent to a corner of another one of the plurality of scallops, the scallops thus being spaced around the bottom of the bottle adjacent to one another.

3. The specimen container of claim 1, wherein the scallops comprise ramp-like indentations.

4. The specimen container of claim 1, further comprising a detent ring formed in the cylindrical wall.

5. The specimen container of claim 1, wherein the specimen container comprises a blood culture bottle.

6. A specimen container for receiving a sample, comprising:
a body having a side wall defining an interior of the body, an upper portion and a base,
wherein the base further comprises a raised rim extending upwards into the interior of the body, the rim defining an interior chamber and an exterior chamber; and
a sensor in the interior chamber or exterior chamber.

7. The specimen container of claim 6, wherein the body defines a central axis, and wherein the raised rim is centered on the central axis.

8. The specimen container of claim 6, wherein the side wall is of cylindrical form, raised rim is circular in from, and wherein the diameter of the raised rim is between 50 and 90 percent of the diameter of the side wall of the body.

9. The specimen container of claim 6, wherein the specimen container comprises a blood culture bottle.

10. The specimen container of claim 6, further comprising a detent ring formed in the cylindrical side wall.

11. A method of manufacturing a specimen container, comprising the steps of:
providing a body having a side wall defining an interior of the body, a top portion and a base, wherein the base further comprises a raised rim extending upwards into the interior of the body, the rim defining an interior chamber and exterior chamber; and
introducing a liquid phase sensor polymer matrix into the interior chamber or exterior chamber and curing the polymer matrix into a solid phase in place.

12. The method of claim 11, wherein the rim is spaced from the side wall.

13. A method of manufacturing a specimen container for receiving a sample, comprising the steps of:
providing a body having a side wall defining an interior of the body, an upper portion and base, the side wall including a transition portion connecting the side wall to the base, wherein the transition portion comprises a plurality of scallops providing indentations in the side wall, the plurality of scallops extending at least partially around the transition portion and extending inwardly toward the interior of the container to reduce the volume thereof; and
introducing a liquid phase sensor polymer matrix into the reduced volume region defined by the plurality of scallops and curing the polymer matrix into a solid phase in place.

14. The method of claim 13, wherein the scallops are formed circumferentially around the entire transition portion.

15. The method of claim 14, wherein the scallops all have substantially the same size and shape.

16. The method of claim 14, wherein the scallops are formed in the body in a non-rotationally symmetric manner.

17. The method of claim 14, wherein the scallops are formed in the body in a rotationally symmetric manner.

18. The method of claim 14, wherein the scallops comprise ramp-like indentations.

19. The method of claim 15, wherein the scallops comprise arcuate-like indentations.

20. The method of claim 15, wherein the cured polymer matrix material functions as a colorimetric sensor.

21. The specimen container of claim 1 wherein the base of the specimen container has an inwardly-extending dome shape.

22. The specimen container of claim 1 wherein said plurality of scallops results in a container having an increased strength or rigidity.
